(19) United States
(12) Patent Application Publication
(10) Pub. No.: US 2018/0200465 A1
(43) Pub. Date: Jul. 19, 2018

(54) DEVICE FOR USE WITH A HUMAN OR ANIMAL BODY

(71) Applicant: Tim Kiefte, Auckland (NZ)

(72) Inventor: Tim Kiefte, Auckland (NZ)

(21) Appl. No.: 15/103,837

(22) PCT Filed: Dec. 12, 2014
(86) PCT No.: PCT/GB2014/053697

§ 371 Date: Jun. 10, 2016

(30) Foreign Application Priority Data
Dec. 13, 2013 (GB) 1322107.2

(51) Int. Cl. A61M 16/04

(52) U.S. Cl. CPC A61M 16/047; A61M 16/0497; A61M 2205/8206; A61M 2205/583; A61M 2205/59; A61M 2205/587

(57) ABSTRACT

A device (20), for use with a human or animal body, comprises a device body (22) including an opening (26). The device body (22) is directly attachable over a stoma of a human or animal body to align the opening (26) and stoma. The device (20) further comprises a light emitting element (40).

This invention relates to a device for use with a human or animal body, a tracheostomy apparatus comprising the device, a method of manufacturing the device, and a method of manufacturing the tracheostomy apparatus.

Treatment of a patient requiring constant access to a cavity within the patient's body may be carried out using a medical device, such as a tracheostomy apparatus, to access the cavity within the patient's body. Such access may be obtained via a stoma of a human or animal body, which is a natural or artificial gap formed in a human or animal body that leads into a cavity within the human or animal body. An example of a stoma is an incision in the skin of the human or animal body.

According to a first aspect of the invention, there is provided a device, for use with a human or animal body, comprising:
a device body including an opening, the device body being directly attachable over a stoma of the human or animal body to align the opening and stoma; and
a light emitting element.

It will be understood that, for the purposes of the specification, the opening may be any structure (e.g. an aperture) that permits access to a stoma of the human or animal body when the device body is directly attachable over the stoma to align the opening and stoma, and that alignment of the opening and stoma includes partial and full alignment of the opening and stoma.

It will be appreciated that attachment of the device body over the stoma of the human or animal body is intended to include, but is not limited to, adhesion and fastening. Such fastening may be carried out using, for example, a support rope, bandage, bracelet, belt or necklace attached to the device body.

The combination of the device body with the light emitting element provides the device according to the invention with an inbuilt illumination mechanism.

The inbuilt illumination mechanism of the device according to the invention makes it easier for another person to visually locate the device as worn by a user, even from afar, in low-light and dark conditions without having to rely on an external light source (e.g. a room light). In addition the external light source might not be readily available due for various reasons, such as a power outage. Moreover, in hospital wards with multiple patients, a nurse may have to check on the user in low-light or dark conditions to avoid waking up other patients.

The ability to visually locate the device in low-light and dark conditions allows another person to easily check whether the device is dislodged from its correct position on the user, and whether the opening is obstructed or blocked, for example, by another part of the user's body, clothing, bedding, food or vomit. This not only provides the other person with peace of mind that the device is in proper working order, but also allows the other person to provide the user with immediate attention if the device is dislodged or its opening is obstructed/blocked. The latter is particularly important when dislodgment of the device, or obstruction/blockage of the opening can endanger the health and life of the user.

Since the stoma leads to a cavity within the human or animal body, it is important to keep the device as sterile as possible during servicing of the device in order to minimise infection of the human or animal body. The inclusion of the inbuilt illumination mechanism makes it easier to attend to the device, for example, when cleaning the device using a sterile tool (e.g. a catheter or suction tool). This is because the inbuilt illumination mechanism makes it easier to visually locate the device, and in particular the opening, in order to carry out any necessary servicing.

Otherwise the lack of an inbuilt illumination mechanism in the device may result in, during attempts to locate the device, use of the sterile tool to accidentally touch other, unsterile parts of the user or nearby objects due to the lack of visibility in low-light or dark conditions. This not only increases the risk of infection, but also makes the servicing process more costly due to the need to replace the tool that is rendered unsterile.

Whilst it is possible to use a separate flashlight or head-mounted light to illuminate the device body, both types of lights results in light being directly shone onto a user's face, which not only might wake up the user but also bring visual discomfort to the user. In addition a flash light is required to be held by a hand at all times, thus leaving only one free hand to clean the device body, while a head-mounted light requires time to set up before it can be used to illuminate the device body.

The light emitting element may be arranged to partially or fully illuminate the device, depending on the requirements of the user.

In embodiments of the invention the light emitting element may be arranged to illuminate at least part of the opening. For example, the light emitting element may be arranged to be located about at least a portion of a perimeter of the opening.

Arrangement of the light emitting element to illuminate at least part of the opening not only improves the ability to visually locate the opening of the device body, but also makes it easier to check whether the opening is obstructed or blocked, even when the user is coughing, spluttering or struggling.

The device body may be a tubular device body. The opening may be defined by an internal bore extending through the tubular device body.

The light emitting element may be arranged to be located on a tubular end of the tubular device body. This permits visual assessment of the tubular end for any obstruction that may prevent air passing into and out of the internal bore.

The light emitting element may be arranged to illuminate the internal bore. This permits ready inspection to identify any blockage of the internal bore.

In further embodiments of the invention the light emitting element may be in the form of a symbol, optionally a direction symbol, a dot, a subtle dot, a letter, a word, a number, a logo and/or a graphic.

Forming the light emitting element as a direction symbol enables straightforward determination of the orientation of the device, thus making it easier to check whether the device is in proper working order or dislodged and whether the opening is obstructed/blocked.

Forming the light emitting element as a dot, or optionally a subtle dot, provides a minimalist approach for arranging the light emitting element on the device to provide the desired visual indication or location without significantly altering the aesthetics of the device.

It will be appreciated that, for the purposes of this specification, the term “subtle” is intended to refer to a shape that is designed to be not easily perceived by the naked eye.

Forming the light emitting element as a letter, a word, a number, a logo and/or a graphic not only permits information to be displayed on the device, but also permits aesthetic enhancement of the device.

Optionally the direction symbol may be arranged to indicate the location of the opening. Formation of the light emitting element in this manner allows straightforward visual location of the opening relative to the rest of the device.

The configuration of the light emitting element may vary depending on specific requirements of the device, such as:
the ability to emit light without being powered by a power source;
the ability to be highly visible in low-light and dark conditions, but minimally visible in well-lit conditions;
the property of being non-radioactive, so that the device body can be attached over the stoma of a human or animal body without causing discomfort to the user or damaging the material(s) of the device.

For example, the light emitting element may include one or more of, but is not limited to:
a fluorescent material, namely a material that is capable of emitting light after absorbing light or other electromagnetic radiation. In use, a light emitting element including a fluorescent material may be permitted to absorb light from daylight or from an artificial light source, so that it is then able to emit light in low-light or dark conditions;
a luminescent material, namely a material that is capable of emitting light without involving heat. Emission of light by a luminescent material may be caused by, for example, electrical energy, subatomic motions or piezoelectric stress;
a phosphorescent material, namely a material that is capable of gradually emitting light after absorbing light or other electromagnetic radiation. The rate of emission of light from a phosphorescent material may vary from a few minutes to several hours depending on the quality and amount of phosphorescent material used;
a soft glow material, namely a material that is capable of producing light that has low glow intensity but is readily visible. Use of a soft glow material in the light emitting element makes it easier on the eyes of a user looking at the light emitting element;
a light reflective material, namely a material that is capable of reflecting light. A light emitting element including a light reflective material is particularly useful in low-light conditions;
a light source, which may be any type of source that is capable of generating its own light.

In still further embodiments of the invention the light source may be a cold light source, and/or the light source may be a light emitting diode (LED), preferably an organic light emitting diode (OLED), which is capable of producing a soft glow that is readily visible. Use of a cold light source as the light emitting element results in minimal or zero heat emission from the device. This allows the device body to be attached over the stoma of a human or animal body without causing discomfort to the user or damaging the material(s) of the device.

In embodiments employing the use of a light source, the device may further include a power source for electrically powering the light source. An example of the power source is a battery. Small, lightweight batteries (e.g. compact lithium batteries that are capable of providing power with minimal heat production) are readily available, and can be incorporated into the device without significantly adding to the cost, size, and weight of the device.

The device may include an optical fibre arranged to guide the light emitted by the light emitting element so as to illuminate a target area or volume of the device. The flexibility of the optical fibre provides additional design options to enable the light emitting element to illuminate a target area or volume of the device, e.g. when the size and/or shape of the light emitting element prevents it from effectively illuminating the target area or volume, while the lightweight nature of the optical fibre means that the use of the optical fibre in the invention does not significantly add to the weight of the device.

The light emitting element may be separate from the device body. In such embodiments the light emitting element may be, but is not limited to:
adhered to the device body;
fastened to the device body, e.g. using a screw or clip; or
applied onto an outer surface of the device body. The device may include a flange attached to the device body, wherein the outer surface forms at least part of the flange.

Application of the light emitting element onto an outer surface of the device body may be, for example, carried out by coating the device body with a suitable coating material.

In other embodiments of the invention the device may include a component that is detachably connected to the device body, the component including the light emitting element. The component may be, but is not limited to, a filter, sleeve or cap.

In this manner the light emitting element and the device body can be detached from each other. This not only allows replacement of the light emitting element if it is damaged, but also permits the light emitting element to be reused if the device body is required to be replaced on a regular basis.

Alternatively the device may include a component that is permanently connected to the device body, the component including the light emitting element. Again, the component may be, but is not limited to, a filter, sleeve or cap.

In still other embodiments of the invention the light emitting element may be integral with the device body. In such embodiments the light emitting element may include, but is not limited to, a pigment, additive or dye material (e.g. a fluorescent pigment, additive or dye material, or a luminescent pigment, additive or dye material).

The shape and structure of the device body may vary depending on specific requirements of the device. For example, the device body may be shaped as a protrusion or a disc, and/or the device body may include a metallic material and/or a polymer material.

The device may be a hub (e.g. a disc-shaped hub) or connector (e.g. a protrusion-shaped connector) for connection to a cannula that may be inserted into a human or animal body.

According to a second aspect of the invention, there is provided a tracheostomy apparatus comprising a device according to any embodiment of the first aspect of the invention.

The combination of a light emitting element and the device body in the tracheostomy apparatus according to the invention provides the tracheostomy apparatus with an inbuilt illumination mechanism that permits use of the device in low-light and dark conditions.

This not only makes it easier to check whether the tracheostomy apparatus is dislodged and whether the opening is obstructed or blocked, all of which could lead to a health- or life-threatening situation, but also makes it easier to service the tracheostomy apparatus, which might require cleaning up to tens or hundreds of times a day.

It will be appreciated that, although the device according to the invention is primarily described in the specification with respect to its use in a tracheostomy apparatus for a human body, it is applicable for use in other types of apparatus, each of which comprises a device body that includes an opening and is directly attachable over a stoma of the human or animal body to align the opening and stoma.

According to a third aspect of the invention, there is provided a method of manufacturing a device, the device comprising a device body including an opening, the device body being directly attachable over a stoma of a human or animal body to align the opening and stoma, the method comprising the step of combining the device body with a light emitting element.

According to a fourth aspect of the invention, there is provided a method of manufacturing a tracheostomy apparatus, the method comprising the steps of: carrying out the method according to the third aspect of the invention; and forming the tracheostomy apparatus from the device manufactured in accordance with the method according to the third aspect of the invention.

A preferred embodiment of the invention will now be described, by way of a non-limiting example, with reference to the accompanying drawings in which:

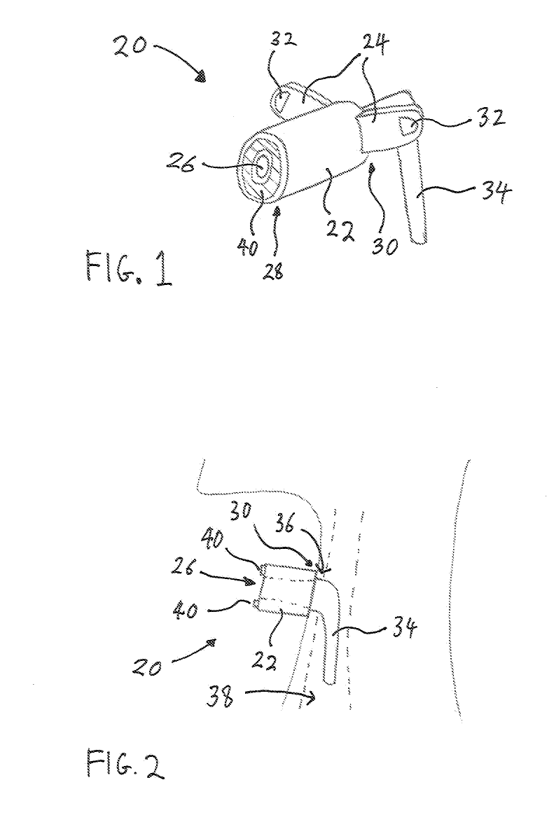

FIG. 1 shows a tracheostomy apparatus according to a first embodiment of the invention;

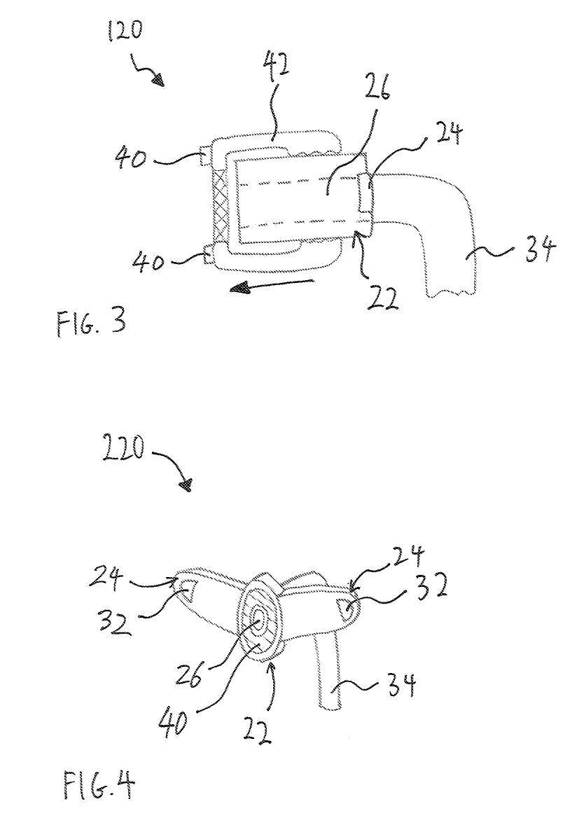

FIG. 2 shows the tracheostomy apparatus of FIG. 1 as worn by a user;

FIG. 3 shows a tracheostomy apparatus according to a second embodiment of the invention;

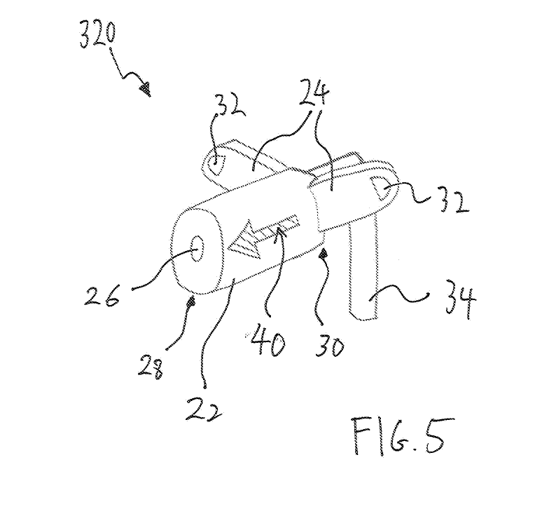

FIG. 4 shows a tracheostomy apparatus according to a third embodiment of the invention; and

FIG. 5 shows a tracheostomy apparatus according to a fourth embodiment of the invention.

A first tracheostomy apparatus 20 according to a first embodiment of the invention is shown in FIG. 1.

The first tracheostomy apparatus 20 includes a connector. The connector includes a device body 22 and a pair of flanges 24.

The device body 22 is shaped as a tubular protrusion with proximal and distal ends 30,28, thus forming a protrusion-shaped connector. The device body 22 includes an aperture 26 extending therethrough between the proximal and distal ends 30,28, whereby the aperture defines an internal bore. The flanges 24 are respectively attached to opposite flanks of the device body 22 located at the proximal end 30 of the tubular protrusion. Each flange 24 includes a hole 32 through which a support rope may pass through.

The first tracheostomy apparatus 20 also includes a cannula 34 connected to the aperture 26 at the proximal end 30 of the device body 22 so as to be in direct fluid communication with the aperture 26.

In use, when the first tracheostomy apparatus 20 is worn by a human user as shown in FIG. 2, the device body 22 is directly attached over a stoma 36 on the neck of the user to fully align the aperture 26 and stoma 36 at the proximal end 30 of the device body 22, and the cannula 34 is inserted into the stoma 36 and into the user's trachea 38. It is envisaged that, in other embodiments of the invention, the device body may be directly attached over a stoma in the user's neck to partially align the aperture and stoma.

The position of the flanges 24 on opposite flanks of the device body 22 at the proximal end 30 of the tubular protrusion allows a support rope to be passed through the holes 32 in the flanges 24 and around the user's neck in order to hold the first tracheostomy apparatus 20 against the user's neck.

The first tracheostomy apparatus 20 further includes a light emitting element 40. In the embodiment shown, the light emitting element 40 is a cold light source in the form of an organic light emitting diode (OLED). The first tracheostomy apparatus 20 further includes a battery (not shown) for electrically powering the OLED.

The light emitting element 40 is arranged to be located about the perimeter of the aperture 26 at the distal end 28 of the device body 22, i.e. at a tubular end of the device body 22. In embodiments of the invention, it is envisaged that the light emitting element may be arranged to be located about a portion of the perimeter of the aperture.

In this manner the device body 22 is combined with the light emitting element 40 to provide the first tracheostomy apparatus 20 with an inbuilt illumination mechanism that is arranged to directly illuminate the aperture 26.

Such an inbuilt illumination mechanism makes it easier for another person to visually locate the first tracheostomy apparatus 20 as worn by the user, even from afar, in low-light and dark conditions without having to rely on an external light source (e.g. a room light). In addition the external light source might not be readily available due for various reasons, such as a power outage. Moreover, in hospital wards with multiple patients, a nurse may have to check on the user in low-light or dark conditions to avoid waking up other patients.

The ability to visually locate the first tracheostomy apparatus 20 in low-light and dark conditions allows another person to easily check whether the first tracheostomy apparatus 20 is dislodged from its correct position on the user, which may occur due to the user's movements while sleeping. The ability to visually locate the first tracheostomy apparatus 20 in low-light and dark conditions also allows another person to easily check and whether the aperture 26 is obstructed or blocked, for example, by another part of the user's body, clothing, bedding, food or vomit.

This not only provides the other person with peace of mind that the first tracheostomy apparatus 20 is in proper working order, but also allows the other person to provide the user with immediate attention if the first tracheostomy apparatus 20 is dislodged or its aperture 26 is obstructed/blocked. The latter is particularly important when dislodgment of the first tracheostomy apparatus 20, or obstruction/blockage of the aperture 26 can endanger the health and life of the user, since the user would not be able to breathe properly of the first tracheostomy apparatus 20 is not in proper working order.

In use of the first tracheostomy apparatus 20, moisture in the user's lungs exits through the aperture 26, and mucus or vomit sometimes also exit through the aperture 26. A sterile tool, such as a catheter or suction tool, is used to clean the aperture 26 and cannula 34 in order to keep the aperture 26 and cannula 34 free of such moisture, mucus or vomit. It may be necessary to carry out such cleaning up to several tens or hundreds of times a day.

Since the aperture 26 and cannula 34 lead to the airways inside the user's neck, it is important to keep the first tracheostomy apparatus 20 as sterile as possible during cleaning of the first tracheostomy apparatus 20 in order to minimise infection of the airways. The inclusion of the inbuilt illumination mechanism makes it easier to clean the aperture 26 and cannula 34 and at the same time keep the tool sterile, because the inbuilt illumination mechanism makes it easier to visually locate the aperture 26 in order to carry out any necessary cleaning with the tool.

Otherwise the lack of an inbuilt illumination mechanism in the first tracheostomy apparatus 20 may result in, during attempts to locate the aperture 26, use of the sterile tool to accidentally touch other, unsterile parts of the user or nearby objects due to the lack of visibility in low-light or dark conditions. This not only increases the risk of infection, but also makes the cleaning process more costly due to the need to replace the tool that is rendered unsterile.

Moreover arrangement of the light emitting element 40 to directly illuminate the aperture 26 not only improves the ability to visually locate the aperture 26, but also makes it easier to check whether the aperture 26 is obstructed or blocked, even when the user is coughing, spluttering or struggling.

The combination of the light emitting element 40 and the device body 22 in the first tracheostomy apparatus 20 therefore provides the first tracheostomy apparatus 20 with an inbuilt illumination mechanism that permits use of the device in low-light and dark conditions. It will be appreciated that the cannula 34 does not necessarily require its own inbuilt illumination mechanism, as its insertion into the user's neck means that it is not visible from the outside.

Furthermore use of an OLED as the light emitting element 40 produces a soft glow that is not only readily visible, but also results in minimal or zero heat emission from the first tracheostomy apparatus 20. This allows the device body 22 to be attached over the stoma 36 in the user's neck without causing discomfort to the user or damaging the material(s) of the device.

Small, lightweight batteries are readily available, and can be incorporated into the first tracheostomy apparatus 20 without significantly adding to the cost, size, and weight of the first tracheostomy apparatus 20.

Optionally the light emitting element 40 may be arranged to illuminate the aperture 26, i.e. the internal bore. More specifically, the light emitting element 40 may be arranged to illuminate the internal bore in order to permit inspection of the internal bore from the distal end 28 to the proximal end 30 of the device body 22. Arranging the light emitting element 40 in this manner not only permits visual assessment of the aperture 26 at the distal end 28 of the device body 22, but also permits ready inspection to identify any blockage of the internal bore. This enables such an inspection to be included as part of a constant care routine for a user of the first tracheostomy apparatus 20, thus providing health benefits for the user.

A second tracheostomy apparatus 120 according to a second embodiment of the invention is shown in FIG. 3. The second tracheostomy apparatus 120 of FIG. 3 is similar in structure and operation to the first tracheostomy apparatus 20 of FIG. 1, and like features share the same reference numerals.

The second tracheostomy apparatus 120 differs from the first tracheostomy apparatus 20 in that the second tracheostomy apparatus 120 includes a filter 42 that is detachably connected to the device body 22, i.e. the filter 42 can be detached from the device body 22. Such a filter 42 may be designed to filter out dust and other airborne contaminants so that they are prevented from entering the airways of the user.

The filter 42 includes the light emitting element 40. Hence, the light emitting element 40 and the device body 22 can be detached from each other. This not only allows replacement of the light emitting element 40 if it is damaged, but also permits the light emitting element 40 to be reused if the device body 22 is required to be replaced on a regular basis, which can be weekly for a tracheostomy apparatus.

Inclusion of the light emitting element 40 in the filter 42 can be particularly beneficial for users that require continuous or near-continuous use of the filter 42 in the second tracheostomy apparatus 120.

It is envisaged that, in other embodiments of the invention, the filter may be replaced by a cap or sleeve.

It is also envisaged that, in other embodiments of the invention, the filter 42 may be permanently connected to the device body 22.

A third tracheostomy apparatus 220 according to a third embodiment of the invention is shown in FIG. 4. The third tracheostomy apparatus 220 of FIG. 4 is similar in structure and operation to the first tracheostomy apparatus 20 of FIG. 1, and like features share the same reference numerals.

The third tracheostomy apparatus 220 differs from the first tracheostomy apparatus 20 in that, in the third tracheostomy apparatus 220, the connector is replaced by a hub. The device body 22 of the third tracheostomy apparatus 220 is shaped as an annular disc instead of a tubular protrusion, thus forming a disc-shaped hub. Shaping the device body 22 as an annular disc allows the device body 22 to be kept as flush as possible with the skin of the user.

A fourth tracheostomy apparatus 320 according to a fourth embodiment of the invention is shown in FIG. 5. The fourth tracheostomy apparatus 320 of FIG. 5 is similar in structure and operation to the first tracheostomy apparatus 20 of FIG. 1, and like features share the same reference numerals.

The fourth tracheostomy apparatus 320 differs from the first tracheostomy apparatus 20 in that the light emitting element 40 of the fourth tracheostomy apparatus 320 is in the form of a direction symbol. More specifically, the direction symbol is in the form of an arrow that is arranged to indicate the location of the aperture 26.

Forming the light emitting element 40 as a direction symbol in this manner enables straightforward determination of the orientation of the fourth tracheostomy apparatus 320, thus making it easier to check whether the fourth tracheostomy apparatus 320 is in proper working order or dislodged and whether the aperture 26 is obstructed/blocked, and also allows straightforward visual location of the aperture 26 relative to the rest of the fourth tracheostomy apparatus 320.

In other embodiments of the invention it is envisaged that, in place of or in addition to the direction symbol, the light emitting element may take the form of a letter, a word, a number, a logo, a graphic and/or any other symbol. Forming the light emitting element as a letter, a word, a number, a logo and/or a graphic not only permits information to be displayed on the fourth tracheostomy apparatus, but also permits aesthetic enhancement of the fourth tracheostomy apparatus.

In still other embodiments of the invention it is envisaged that, in place of or in addition to the direction symbol, the light emitting element may take the form of a dot or a subtle dot.

The configuration of the light emitting element in each of the described embodiments may vary depending on specific requirements of the respective tracheostomy apparatus, such as:
the ability to emit light without being powered by a power source;
the ability to be highly visible in low-light and dark conditions, but minimally visible in well-lit conditions;
the property of being non-radioactive, so that the device body can be attached over the stoma in the user's neck without causing discomfort to the user or damaging the material(s) of the device.

The light emitting element may be arranged to partially or fully illuminate the tracheostomy apparatus.

For example, the light emitting element may include one or more of, but is not limited to:
a fluorescent material;
a luminescent material;
a phosphorescent material;
a soft glow material;
a light reflective material;
another type of light source, which may be any type of source that is capable of generating its own light.

The light source may be another type of cold light source and/or another type of light emitting diode.

The tracheostomy apparatus may include another type of power source, other than a battery, for electrically powering the light emitting element.

The tracheostomy apparatus may include an optical fibre arranged to guide the light emitted by the light emitting element so as to illuminate a target area or volume of the tracheostomy apparatus, such as the internal bore of the tracheostomy apparatus.

The light emitting element may be separate from the device body. In such embodiments the light emitting element may be, but is not limited to:
adhered to the device body;
fastened to the device body, e.g. using a screw or clip; or
applied onto an outer surface of the device body, which may form at least part of one of the flanges.

Application of the light emitting element onto an outer surface of the device body may be, for example, carried out by coating the device body with a suitable coating material.

It is envisaged that, in other embodiments of the invention, the light emitting element may be integral with the device body. In such embodiments the light emitting element may include, but is not limited to, a pigment or dye material.

Other than a tubular protrusion and an annular disc, the device body may be shaped in other ways depending on specific requirements of the device.

The structure of the device body may vary depending on specific requirements of the tracheostomy apparatus. For example, the device body may include a metallic material (such as stainless steel) and/or a polymer material (such as silicone).

1. A device, for use with a human or animal body, comprising:
a device body including an opening, the device body being directly attachable over a stoma of the human or animal body to align the opening and stoma; and
a light emitting element.

2. A device according to claim 1 wherein the light emitting element is arranged to at least one of: partially illuminate the device, fully illuminate the device, illuminate at least part of the opening, and be located about at least a portion of a perimeter of the opening.

3. (canceled)

4. (canceled)

5. A device according to claim 1 wherein the device body is a tubular device body, the opening being defined by an internal bore extending through the tubular device body.

6. A device according to claim 5 wherein the light emitting element is at least one of: arranged to be located on a tubular end of the tubular device body, and arranged to illuminate the internal bore.

7. (canceled)

8. A device according to claim 1 wherein the light emitting element is at least one of: in the form of a symbol, a direction symbol arranged to indicate the location of the opening, a dot, a subtle dot, a letter, a word, a number, a logo, and a graphic.

9. (canceled)

10. A device according to claim 1 wherein the light emitting element includes one or more of:
a fluorescent material;
a luminescent material;
a phosphorescent material;
a soft glow material;
a light reflective material;
a light source;
a cold light source;
a light emitting diode; and
an organic light emitting diode.

11. (canceled)

12. (canceled)

13. A device according to claim 6 further including any one of: a power source for electrically powering the light source, and a power source in the form of or including a battery for electrically powering the light source.

14. A device according to claim 1 including an optical fibre arranged to guide the light emitted by the light emitting element so as to illuminate a target area or volume of the device.

15. A device according to claim 1 wherein the light emitting element is any one of: separate from the device body, and integral with the device body.

16. A device according to claim 1 wherein the light emitting element is:
adhered to the device body;
fastened to the device body; or
applied onto an outer surface of the device body;

17. A device according to claim 16 including a flange attached to the device body, wherein the outer surface forms at least part of the flange.

18. A device according to claim 15 wherein the light emitting element is separated from the device body, and the device further includes a component that is detachably or permanently connected to the device body, the component including the light emitting element.

19. A device according to claim 18 wherein the component is a filter, sleeve or cap.

20. (canceled)

21. A device according to claim 20 wherein the light emitting element is integral with the device body, and the light emitting element includes a pigment, additive or dye material.

22. A device according to claim 1 wherein the device body is shaped as a protrusion or a disc.

23. A device according to claim 1 wherein the device body includes at least one of: a metallic material, and a polymer material.

24. A device according to claim 1 wherein the device is a hub or connector for connection to a cannula that may be inserted into a human or animal body.

25. A tracheostomy apparatus comprising a device according to claim 1.

26. A method of manufacturing a device, the device comprising a device body including an opening, the device body being directly attachable over a stoma of a human or animal body to align the opening and stoma, the method comprising the step of combining the device body with a light emitting element.

27. A method of manufacturing a tracheostomy apparatus, the method comprising the steps of: carrying out the method of claim 26; and forming the tracheostomy apparatus from the device manufactured in accordance with the method of claim 26.

28. (canceled)

29. (canceled)

30. (canceled)
